(19) United States
(12) Patent Application Publication
(10) Pub. No.: US 2017/0231793 A1
(43) Pub. Date: Aug. 17, 2017

(54) NEUROARTHROMYOFASCIAL ENHANCEMENT WEAR

(71) Applicant: Granite Sports Institute, LLC, Surprise, AZ (US)

(72) Inventors: Aaron PARR, Surprise, AZ (US); Jason ROBERTS, Surprise, AZ (US); Duslin VISSERING, Surprise, AZ (US)

(21) Appl. No.: 15/504,284

(22) PCT Filed: Aug. 17, 2015
(86) PCT No.: PCT/US15/45554

§ 371 Date: Feb. 15, 2017

Related U.S. Application Data
(60) Provisional application No. 62/038,169, filed on Aug. 15, 2014.

(51) Int. Cl. A61F 5/01; A61F 5/03

(52) U.S. Cl. CPC A61F 5/0104; A61F 5/03

(57) ABSTRACT

A garment for enhancing neuroarthromyofascial performance in an individual includes fabric tension lines positioned within the garment in order to align with certain muscle groups of the individual wearing the garment. The fabric tension lines have an elasticity that imparts a force from one end of the fabric tension line to the other. This force transfers to the garment as a whole, and subsequently to the individual. The force pulls one or more joints of the individual into an abnormal alignment, causing a neurological cue in the individual to recruit targeted muscles that restore the proper alignment of the joint. The strength and performance of the targeted muscles is thereby improved. The garment may additionally have compressive properties that have a therapeutic effect on the individual. Methods for making the garment and for using the garment in a physical therapy environment are also disclosed.

### CROSS-REFERENCE TO RELATED APPLICATIONS

This application claims the benefit of U.S. Prov. Pat. App. Ser. No. 62/038,169, entitled “Neuroarthromyofasical Enhancement Wear,” filed on Aug. 15, 2014, and incorporated in its entirety herein by reference.

### BACKGROUND OF THE INVENTION

The body is made of fascial planes and slings intertwining and connecting throughout. No longer is anatomy a singular statement of location; our design gives anatomical location no bearing unless it interacts with what is above, below, and to the sides. The physiologic response within a muscle from elongation, compression, and tension all provide feedback and feedforward mechanisms with the surrounding neuroarthromyofascial tissue. The communication is dynamic, forming both monosynaptic and multisynaptic loops; the key is that nothing is mutually exclusive.

Skilled movement, which by definition is considered coordinated hand, elbow, shoulder, and trunk activity while manipulating an object, would not be possible unless oxygen was able to flow through the tissues to support metabolic function. Diaphragmatic activity, which oxygenates the blood, is thus an elemental/fundamental form and part of movement. The patterns that our body develops over time must incorporate breathing to make the movement patterns efficient/stabilized and fatigue resistant.

Imagine the body and the movement patterns functioning like a pyramid. Each joint required for completion of the movement has their own block within the pyramid. The more complex the movement, the more blocks there are. The foundational block is the core, or trunk, made up of multiple vertebrae. Many muscles are associated with the core and the activity of these muscles help determine how movement away from the core is performed. The core, by both anatomical location and functional definition includes the diaphragm. If all movements are founded upon a “core”, then breathing pattern and dynamic stability are very important. To put this into perspective, respiration occurs around 12 to 15 breaths per minute, or rather, 17-20,000 times per day and increasing even more during exercise, with levels reaching around 35 to 45 breaths per minute. This statistic means the body must integrate breathing 17,000+ times per day into daily posturing or motion. The body's attempts to integrate and coordinate movements become very complex and require an algorithm.

The natural coordinating algorithm includes—and, it is theorized herein, can be influenced by modulating (e.g., via cortex modulation)—central pattern generators (CPGs). CPGs are neuronal circuits that can produce rhythmic motor patterns without sensory or time specific descending input. Before CPGs, movement was thought to occur by reflexes, which were ultimately dictated by afferent input, or rather, sensation. This theory was one of a few recognizable interpretations on how the motor system may operate; others include the hierarchical, systems, and ecological theories. Each theory provides essential insight into answering the question “How do we move?”, but each fail to encapsulate the “How” fully. For example, though reflexes are a large component to the nervous system's circuitry, and represent an early adaptation of the nervous system's response to the changing environment, the reflex theory fell behind CPGs for being considered the base of movement patterns.

The study of fictive motor patterns help to identify what is the base of movement and what are base neurological modulators, which ultimately help refine action and produce skill. This can be observed by isolating the neurological system in saline and bathing it in neural modulators that are normally present from descending neural input. The neuromodulators recruit the stored patterns/circuits involved with movement, which reveal CPGs' inherent circuitry and reliance on descending input for refining motion.

The task of refining motor pool activity is not just from descending neural input but also from peripheral sensory input. The reliance on sensory and descending input is emphasized during activation of a deafferented nervous system. When a deafferented nervous system is activated it produces similar activity patterns to the intact (i.e., afferented) system, but on closer inspection the activity patterns can show differences in rhythm, duty cycle, or phase relationships among the elements. It is important to note these differences change between patterns because some patterns, or rather behaviors, require more or less sensory input. For example, a skilled activity requires more input for coordinated movement than an inherent pattern, such as ambulation. CPGs without descending or sensory information will produce a result that resembles a suitable end product but that lacks specificity.

The depolarization of CPG neurons, and what is involved in the neuronal network, reveal a lot about CPGs in general. Typically, CPGs are pre-motor interneurons driving efferent muscle by activating alpha neuron cells. Despite this being a general rule, many parameters may alter the pre-motor activity. The first is whether the pre-motor cell depolarizes as a “pacemaker” neuron, which would be intrinsically rhythmic, or as a “half-center oscillator”, which has two neurons inhibiting each other reciprocally. The “half-center oscillator” relies on the reciprocating inhibition/excitation states; one neuron's inhibition state leads to the other becoming released from its inhibition and fire. After firing, the cell dips below its spike threshold, releasing its inhibitory effect on the opposite neuron. The “half-center oscillator” is thought to be central for ambulation.

In general, CPGs are structured connections that allow variance and can be influenced by many different routes. They may depolarize a pattern of movement themselves, as with breathing, or may require descending/afferent information to produce skill within the extremities. To understand the complexity and design of skilled movement, evidence suggests neurons may not be inherent strictly to one CPG circuit but may involve multiple circuits that interact. For example, neurons involved with breathing may also be involved with gasping. Evidence furthering the nervous system's “interaction” comes from a study regarding swimming leeches, which show the alternating “wave of movement” coming not from a single CPG but from ascending and descending coupling to produce coordinated CPG activity. This evidence has been suggested to infer what occurs with multi-joint posture/movements, which is coordinated CPGs' dictating posture/motion. In fact, in mudpuppies, it has been shown that antagonists of the same joint activate secondary to coordinated activity from separate CPGs. The coordinated activations are further strengthened by evidence within the stick insect, which show inter-joint activity stems from central coupling and sensory feedback acting upon the CPGs.

The neuroarthromyofascial system in the normal human being is asymmetrical. As our body develops we develop asymmetrically, which makes sense because a majority of our tasks are asymmetrical in nature. Whether we are writing, throwing a ball, kicking, running, walking, eating, or sleeping we are asymmetrical. Being asymmetrical develops from a number of different reasons. One of these reasons is secondary to our body requiring a shift of the center of mass to stabilize the body over our center of pressure for stability, or rather, to position the body's center of mass to create a lever for the body to recruit eccentrically and “fall over itself” into movement. So, the first reason we are asymmetrical is because we are upright beings and need weight shifting to produce both passive and active motion.

The second reason we are asymmetrical is because of our development within the uterus and during our growth. The central tendon of the diaphragm develops at week 4, the diaphragm inserts on L1 at week 8, and the peripheral parts form between weeks 9 through 12. The liver, which sits directly under our diaphragm, starts its development at week 3. By week 6 the liver begins to produce blood cells through hematopoiesis and is a relatively large size weighing about 10% of fetal body weight by week 9. Secondary to the disproportionate size of the fetal liver, and the location under the right diaphragm, the right diaphragm has to recruit more than the left to push the liver inferior and allow diaphragmatic descent. This asymmetrical development of the right diaphragm verse the left is established in the fetus secondary to phrenic nerve excitation of the diaphragm to allow perinasal fluid flow.

Upon analysis of the diaphragm, the central tendon, and its attachment sites, a few different conclusions may be made, which then relate to how the body compensates, or rather, orients itself from the asymmetrical muscle build. The larger right diaphragmatic crus leads to an overdevelopment of the left psoas muscle. This is why the primary population has the lumbar spine in right rotation and thoracic in left rotation. The statistics of L2 orientation in the transverse plane has 64 percent of people with right rotation. Note that 64% is far away from 100% and the body's ability to adapt to internal and external forces are impressive. No two people are the same structurally following tissue adaptation as an infant, adolescent, or adult; also, no two people exhibit the same external stresses each and every day. Monitoring these daily stresses can be helpful in determining the different types of tissue adaptation and abnormal movement mechanics that may have developed over time.

The concept of asymmetry is not new, but also is very theoretical. Identifying the body's development and behaviors leading to asymmetry are presented in a manner that may mislead. In reality it is not one thing, but many, which contribute to and cause the asymmetries we experience.

Products have previously been developed to restore posture by bringing an individual into proper positioning passively. The functional use from the present design provides the opposite approach, which is to actively recruit into proper posture to improve a person's biomechanics, proprioception, and neuroarthromyofascial stability. Those using the present design will achieve not only sports specific goals, but also health goals.

### SUMMARY OF THE INVENTION

The present neuroarthromyofasical enhancement wear (NEW) is a sportswear garment to be used in conjunction with research based training to enhance the recruitment of proper synergistical muscle activity and to reconfigure CPG and/or movement patterns to optimize movements and/or alleviate abnormal conditions. The research based tension lines developed within the sportswear provide torque into abnormal postures. The neurological sensitivity from abnormal posturing requires recruitment of muscles opposing the tension lines. This neurological response restores centration of the joint and allows agonist/antagonistic synergistical activity during sport specific movements.

The NEW garment provides neuroarthromyofascial activation of stabilizing muscles via the fabric tension lines within the garment design. Key stabilizing muscles, for optimal static and dynamic movement, will be focused on to modify and enhance muscle recruitment patterns. This product's design is to be worn during both acute training sessions and chronic postural positioning. The garment will bring an individual's joint centration into abnormal alignment, which will cause neurologic cueing to utilize muscles for the prevention of deviant kinematics. The garment will have anchors upon which tension is established within the fabric, which will then provide the force needed for an individual to recruit appropriate muscle stabilizers.

In one implementation, the present disclosure provides a garment having a tension line formed of an elastic material and having an anchor point at a first end of the tension line and an insertion location at a second end of the tension line, the elastic material being under an elastic tension that pulls the elastic material from the insertion location toward the anchor point, and the anchor point and the insertion location being positioned with respect to a muscle of a wearer of the garment such that, when the garment is worn by the wearer, the garment promotes active neuroarthromyofascial recruitment of the muscle, a CPG pattern, or a movement pattern, by bringing a centration of a joint of the wearer into abnormal alignment such that the wearer must activate the muscle to resist the elastic tension and return the joint to proper position. The garment further includes clothing material surrounding the tension line and giving the garment a wearable shape. The anchor point may be antagonistic to (e.g., opposite) an anatomical insertion of the muscle, and the insertion location may be antagonistic to (e.g., opposite) an anatomical anchor of the muscle. The elastic material may apply a level of compression to the muscle that further promotes active neuroarthromyofascial recruitment of the muscle. The elastic tension and the level of compression may be selected according to one or more performance variables specific to the user. The elastic tension may be selected to provide a resistance that facilitates proper activation of one or more of: the muscle, a first muscle group containing the muscle, a second muscle group not containing the muscle, a CPG pattern, and a movement pattern.

In another implementation, the present disclosure provides a garment including one or more fabric tension lines each having a first end and a second end, and each further having an elasticity that pulls the second end toward the first end, the fabric tension lines being positioned such that, when the garment is worn by a wearer, the fabric tension lines create torque that directs the wearer into an abnormal position. The fabric tension lines may bring one or more joints of the wearer into an abnormal alignment, such as one that causes, in the wearer, a neurologic cueing to activate one or more stabilizing muscles of the wearer that can restore centration of the one or more joints. The fabric tension lines may further be positioned such that the fabric tension lines promote active neuroarthromyofascial recruitment of one or more muscles of the wearer to resist the elasticity of the fabric tension lines and return the wearer to a normal posture.

The fabric tension lines may create the torque when the wearer is performing dynamic movements. The fabric tension lines may additionally or alternatively create the torque when the wearer is not performing dynamic movements. The garment may further include clothing material attached to or integrated with the fabric tension lines and giving the garment a wearable shape. The garment may be a sportswear that fits over a region of the wearer, the region selected from the group comprising: a scapulo-thoracoabdominal region, a lower extremity, a foot, an ankle, a cervical region, and an upper extremity. One or both of the clothing material and the fabric tension lines may apply a level of compression to the wearer to improve activation of one or more muscles in the region over which the garment fits. The garment may be a shirt covering a scapulo-thoracoabdominal region of the wearer, in which shirt the second end may be on the front of the shirt, the first end may be on the back of the shirt, and a first fabric tension line may be positioned to promote active neuroarthromyofascial recruitment of one or more of the wearer's rhomboid muscle group, lower trapezius, and serratus anterior.

In another implementation, the present disclosure provides a method of making a neuroarthromyofascial enhancement garment. The method includes determining a recommended elasticity for an individual, creating one or more fabric tension lines having the recommended elasticity and having a first end and a second end, the elasticity pulling the second end toward the first end, forming a clothing material into a shape that fits over a region of the individual containing one or more targeted muscles, and incorporating the one or more fabric tension lines into the clothing material to form the garment, the one or more fabric tension lines being positioned such that, when the garment is worn by the individual, the one or more fabric tension lines create torque that directs the individual into an abnormal position that causes, in the individual, a neurologic cueing to activate one or more of the one or more targeted muscles. Determining the recommended elasticity for the individual may include determining a value for each of one or more system variables pertaining to the neuroarthromyofascial system of the individual, determining a value for each of one or more performance variables related to the targeted muscles, and determining the recommended elasticity from the values of the one or more system variables and the values of the one or more performance variables. Determining the recommended elasticity for the individual may further include determining, in the individual, one or more fascial connections for the one or more targeted muscles. Incorporating the one or more fabric tension lines into the clothing material may include positioning the first end and the second end of a first of the one or more fabric tension lines such that the first end of the first fabric tension line is directed toward an insertion of a first of the targeted muscles, and the second end of the first fabric tension line is directed toward an anchor of the first targeted muscle.

In yet another implementation, the present disclosure provides a method of promoting active neuroarthromyofascial recruitment of targeted muscles, CPGs, or movement patterns in an individual. The method includes identifying the targeted muscles, CPGs, or movement patterns, determining a value for each of one or more variables related to the targeted muscles, CPGs, or movement patterns, selecting a garment configured to activate the targeted muscles, CPGs, or movement patterns according to the values of the one or more variables, the garment having one or more tension lines each having a first end and a second end, having an elasticity that pulls the second end toward the first end, and being positioned such that, when the garment is worn by the individual, the one or more fabric tension lines create torque that directs the wearer into an abnormal position. The variables may include system variables that identify properties of the targeted muscles and/or surrounding joints, muscles, and fascia. The variables may additionally or alternatively include performance variables that identify intended corrections to movement, stability improvements, CPG pattern alterations, and other measures indicating successful treatment of the targeted muscles. The method further includes causing the individual to wear the garment for a wear time determined from the values of the system and/or performance variables.

### BRIEF DESCRIPTION OF THE DRAWINGS

The present invention will become more apparent from the detailed description set forth below when taken in conjunction with the drawings, in which like elements bear like reference numerals.

FIG. 1 is a diagram of the anterior aspect of a NEW shirt in accordance with the present disclosure.

FIG. 2 is a diagram of the posterior aspect of a NEW compression pant in accordance with the present disclosure.

FIG. 3 is a diagram of the anterior aspect of the compression pant of FIG. 2.

FIG. 4 is a diagram of the left side of the compression pant of FIG. 2.

FIG. 5 is a diagram of the anterior aspect of a NEW sleeve in accordance with the present disclosure.

FIG. 6 is a diagram of the anterior aspect of a NEW hood in accordance with the present disclosure.

FIG. 7 is a diagram of the posterior aspect of the hood of FIG. 6.

FIG. 8 is a diagram of the left side of the hood of FIG. 6.

FIG. 9 is a diagram of the medial aspect of a NEW sock in accordance with the present disclosure.

FIG. 10 is a diagram of the lateral aspect of the sock of FIG. 9.

### DETAILED DESCRIPTION

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures. The figures depict selected embodiments and are not intended to limit the scope of embodiments of the invention. Skilled artisans will recognize the examples provided herein have many useful alternatives and fall within the scope of embodiments of the invention.

The following description refers to elements or features being “connected,” “attached,” or “coupled” together. As used herein, unless expressly stated otherwise, these terms mean that one element/feature is directly or indirectly connected, attached, or coupled to another element/feature, and not necessarily mechanically. Likewise, unless expressly stated otherwise, “coupled” means that one element/feature is directly or indirectly coupled to another element/feature, and not necessarily mechanically, such as when elements or features are embodied in program code. Thus, although the figures depict example arrangements of processing elements, additional intervening elements, devices, features, components, or code may be present in an actual embodiment.

The various aspects of the invention will be described in connection with providing a physically therapeutic treatment for a subject by applying a garment that enhances neuroarthromyofascial activity. That is because the features and advantages that arise due to the invention are well suited to this purpose. However, it should be appreciated that the invention is applicable to other procedures and to achieve other objectives as well.

The design of the garment follows research driven myofascial connections and nervous system modulation. The garment will influence the neurological system by altering the sensory input into the cortex, for descending modulation, and at the level of the spinal cord, for interneuronal “reflexive” modulation. Despite these being two main pathways for CPG modulation they are not inclusive of all the opportunities the garment has on the neuroarthromyofascial system. Altering cognitive processing secondary to comfort of compression, altering fibroblastic differentiation and myofibroblast processing, altering sympathetic activity, and vascular flow are just a few of the other benefits.

In addition to the focus on neurological modification, the garment may modify the muscular, fascial, and arthro systems. The strength and integrity of these systems are not mutually exclusive. Muscle, fascia, bone, cartilage hypertrophy, protein synthesis, mitochondrial modification, substrate concentrations, etc., are all part of the body enhancement the garment provides. “Muscle,” as used herein, is therefore not strictly singular and anatomic, but refers to the complex process regarding movement and is an end product of CPG and movement pattern modulation. Prolonged exposure (i.e., wearing) to the garment will produce remodeling of the neuroarthromyofascial systems and allow proper function.

The present NEW garment contemplates any garment that uses tension lines to promote active neuroarthromyofascial recruitment into proper position during both static and dynamic movements. Detailed below and illustrated in the Figures are several designs for garments that fit over various regions of an individual, but the NEW paradigm is ever-evolving and its concepts can be applied to individual muscles and/or joints, muscle groups, and regions of any size, including without limitation lower extremity, foot/ankle, cervical, and upper extremity recruitment modulation. These designs effectively utilize the inter-dependent relationships within the body to promote optimal performance. By both taking into account dense connective tissue lines coupled with joint specific and activity specific muscles during the placement of the tension lines, the garment will help to optimally recruit myofascial patterns and modify afferent activity to improve isometric or dynamic stability. This gives the garment the wide functional capability of influencing abnormal postures and both generic and patient specific muscle coordination deficits. For instance, a patient may benefit from tension lines causing scapula retraction if the internal rotators were lacking force during the throwing motion.

The garments described below include fabric tension lines that are unique to the garment design. A fabric tension line is a strip or panel of elastic material having a length and a width and an elasticity. An anchor point at one end and an insertion location at the other end will be used to direct the elastic tension of the fabric, which will pull from insertion to anchor. The anchor and insertion may be opposite a muscle's anatomical anchor/insertion secondary to the concept of promoting active proper muscular recruitment patterns. This concept is in direct contrast to existing postural garments, which base their design on bringing an individual into proper position passively, which is then hypothesized to carry over into maintaining the position from neural feedback loops after the garment is taken off. The fabric tension lines are integrated with or attached to clothing material that forms the rest of the garment. The fabric tension lines and the clothing material may have compressive properties that further enhance muscle recruitment.

Before dissecting the fascial network and the intertwined nervous system, it is important to identify the body strictly from an anatomical standpoint. The body develops systematically and more or less in proportion to another. It is easy to point out discrepancies, such as trunk height verse lower extremity length, but studies have shown that in general, people prescribe to a norm. A key anatomical feature that applies to all individuals, and is focal to the present garment and its use, is the myofascial complex and its delineation into myofascial lines. Following is an explanation of the myofascial complex, derived from Anatomy Trains: Myofascial Meridians for Manual & Movement Therapists, Third Edition, by Thomas W. Myers. There are seven main myofascial interconnected relationships, or “trains,” in the body, all of which may be altered by wearing one or more of the present garments. The trains include the superficial back line (SBL), superficial front line (SFL), lateral line, spiral line, arm lines, functional lines, and deep front line (DFL).

Each individual may either over-utilize or under-utilize a train; it is important to understand why deficits exist between the active and inert tone within these trains. The myofascial complex is important for understanding human motion and posture. The body becomes a structure dependent on tension with concepts surrounding biotensegrity. Tensegrity is essentially defined as isolated components in compression inside a net of continuous tension, in such a way that the compressed members (usually bars or struts) do not touch each other and the prestressed tensioned members (usually cables or tendons) delineate the system spatially. This means that the body relies on the inert muscle and fascia tissue, working in synchrony, to promote an upright posture.

The SBL supports the body in a continuous and ipsilateral manner from the occiput to the plantar surface of the feet. The primary muscles/inert tissue within the myofascial construct would be the epicranial fascia, erector spinae, thoracolumbar fascia, sacrotuberous ligament, hamstrings, triceps surae complex, and plantar fascia. Important to any myofascial train, such as the SBL, is identifying how it may interact physically with the peripheral nervous system. For example, the SBL may require analysis when an individual lacks proper postural positioning by deviating into kyphosis, or rather, a position anterior to the coronal plane of the body. If a patient is having radicular symptoms into the lower leg it may be appropriate to check how the sciatic nerve relates to the SBL. The biceps femoris' close association with the sciatic nerve can tension and/or compress the nerve. This may cause peri-neural connective tissue referral patterns or true radiculopathic pain secondary to axonal compression. Radiculopathy is important to analyze in all areas of the body because many entrapment sites exist, which ultimately alters tissue sensitivity thresholds and subsequent CPG activity. Sensitization can either occur from an increase in muscle tone over the nerve tissue or from possible trigger point development. Abnormalities related to the superficial back line may not be strictly tightness, but may include a disconnect between adjacent joints, with hypermobility occurring to remedy another joint's hypomobile status. If this is the case, and the person is not strong enough to stabilize a hypermobile joint, intervention is required.

The present garment may decrease trigger points and compression on some of the more common entrapment sites, such as the piriformis. To reduce the compression, multiple concepts will be employed, such as antagonistic inhibition and/or facilitation to inhibited and lengthened tissue typically antagonistic of the entrapment site. Antagonistic inhibition is a neural reflex within the spinal cord causing muscle inhibition from the contraction of a muscle's antagonist. So, for instance, the piriformis may have a reduction in tone if a desk worker is wearing the garment during sitting: the fabric tension lines cause the nervous system to recruit the antagonistic muscle complex, which are the adductors. Fabric tension lines may recruit muscles surrounding hypermobile joints, for active stabilization, which over time will promote coordinated inter-articular motion and remodel myofascial tissue for larger passive viscoelastic forces. If the garment is being used to stabilize hypermobility, such as the erector spinae in the SBL, it is important to determine what kind of excess motion exists. Abnormal posturing is one type of hypermobility caused by tissue remodeling of both inert and muscular tissue in either a shortened or lengthened position. For instance, kyphotic posture, and the degenerated or lax SBL associated therewith, requires active cuing through the garment to contract myofascial structures closely associated and invaginated in the fascia for a more stable upright posture.

The SFL works to counter the SBL. The SFL has a higher proportion of type I muscle fibers, which lends itself to stability. The muscles involved, from distal to proximal, are to include the tibialis anterior, quadriceps, rectus abdominus, pectoralis, and sternocleidomastoid. This fascial train is involved in both posture and dynamic mobility with posture restrictions typically limiting proper myofascial activity in the other trains, such as the superficial back line during gait or running. An example of this limitation can stem from quadriceps tightness, which influences pelvic positioning and ability to perform terminal stance secondary to diminished hip extension. Further, if the quadriceps are tight, and the pelvis tilted anterior, one may have pathologic gluteal or hamstring tissue lengthening. The garment may remedy this issue, and create a more neutral pelvis, by facilitating even greater anterior pelvic tilt using tension lines anterior to the pelvis, which will stimulate gluteal and hamstring activity to overcome the passive elastic forces of the quadriceps.

The lateral line is a myofascial train running down the lateral part of the skeleton. Secondary to a human's base of support existing primarily in the frontal plane, the lateral line exists to stabilize an individual and accelerate/decelerate. Tightness in this train may affect an individual's ability to perform, not only in a coronal but also sagittal and transverse plane. If the lateral line is too tight, such as in the IT-band, the adductors may not be able to be in good position to stabilize the femur on the pelvis. This discrepancy in stability may lead the body to develop compensations within the trunk and foot, or upper extremity, to improve sagittal plane motion. The garment may be designed to stimulate the adductor tissue to overcome the tightness of the lateral line. Wearing the garment while engagement in an active stretching program may further promote stability in the ranges gained.

The spiral line is essential in stabilizing the body and promoting movement through rotation. Since most of the body's motions are asymmetrical, the spiral line is constantly active. In the upper body, the spiral line connects the splenius capitis and cervicis with the rhombo-serratus complex on the opposite side of the spine, then extends into the external oblique and opposite internal oblique. In the lower body, the spiral line extends from the ASIS, where the upper body spiral line ends, into the TFL, iliotibial tract, tibialis anterior, fibularis longus, biceps femoris, sacrotuberous ligament, erector spinae and into the epicranial fascia. The spiral line's complexity leads to many issues that may develop. Three common discrepancies, between the two spiral lines propagating in the body, concern abnormal ratios regarding myofascial strength, passive elastic forces, and muscle length. If asymmetry is seen between the two lines, rotation is most commonly noted. For instance, if the spiral line originating from the left occiput is more taut as compared to the right then excessive left thoracic rotation and right pelvic rotation may occur. This may create a disparity in posture that causes the body to compensate. The garment can help remedy the compensations or abnormal baseline posturing by passively bringing a person into left thoracic and right lumbar rotation further, thus causing recruitment of the thoracic and lumbar to a more neutral posture.

The arm lines are dissected into four layers: superficial front arm line, superficial back arm line, deep front arm line, and deep back arm line. These lines are not functional unless they work off of a stable scapula-thoracic region. It is important to identify potential trunk dysfunction before trying to remedy issues within the arm lines and other trains specific to the upper extremity. The kinematics within the upper extremity are complex and the interaction of the triplanar joints with four fascial lines contribute to the complexity. Often, because of the proximity of each upper extremity compartment related to the other, tightness in one train will cause abnormal tension in an other. An example of this relation can be seen during manual techniques resolving pain within the lateral extensor tendon complex by myofascially releasing the medial compartment. Thus, it is important to identify an individual's baseline pattern of movement and how each train may relate to the completion of that movement. The garment may then be applied to improve function in the upper extremity by activating neurologically inhibited trains, blocking excessive motion with tension lines, and promoting antagonistic stabilization, which will help decrease pathologic repetitive stress.

The functional lines are not considered postural myofascial trains as evidenced by their grounding in phasic muscles, such as the pectoralis, latissimus dorsi, glute max, adductor longus, sartorius, and hamstring tissue. Three functional lines are said to exist: the back, front, and the ipsilateral functional line. Because many movements require the “core” trains to precede and stabilize functional patterns, wearing a garment associated with the functional lines may be suitable for already demonstrating good stability. Once again, identification of functional movement characteristics is essential for the advancement of proper function using the garment.

In contrast to the functional lines, the deep front line (DFL) is a “core” related train. This train incorporates the diaphragm and is essential in stabilizing the body in a tri-planar world. Other key muscles involved within the train include the longus colli, hyoid, scalene, masseter, and temporalis muscles superior to the diaphragm, and the transversus abdominus, psoas, quadratus lumburum, iliacus, pelvic floor, hip adductors, posterior tibialis, flexor halluces longus, and flexor digitorum longus below the diaphragm. If an individual demonstrates inability to support an unstable pelvis or lumbar spine, research indicates they may respond to a stabilization program. Facilitation of proper CPG circuitry, to stabilize the pelvolumbar region, will include stimulating combined muscle activity for the pressurization of the abdominal “fluid ball.” As the diaphragm descends, the visceral contents become forced into the pelvic floor and sides of the abdominal complex. This force stimulates a contraction of stabilizing muscles, but if a certain component to the complex, such as the pelvic floor and adductors are not recruiting, the fluid ball looses its pressure and the force descends out the inferior component. The present garment can help an individual with muscle activity loss to any part of the “fluid ball.” For instance, if the pelvic floor is the weak link, the adductors will be stimulated by the garment to then elicit a pelvic floor contraction and provide reciprocal force to the diaphragm and gravity pushing the abdominal contents inferiorly. The fabric tension lines in this instance will passively pull an individual into abduction to facilitate the adductor contraction.

Despite the fascial lines having a predominance of certain muscle fiber types, it does not mean every muscle in that train shares this proportion. Often type I muscles, which are considered postural, are interconnected in the chain with type II muscles, considered phasic. This interplay and between muscles change among people with different movement demands. Some individuals may train strictly type I fiber function, either through exercise or daily postural demands, which can create asymmetries in a singular fascial train. If there are discrepancies in strength or endurance within a fascial train, and certain muscles need to make up for others to provide stability, then abnormal arthromechanics and pathologic motion will occur. It will be important to assess myofascial trains specific to an individual's economy requirement and neural recruitment sequence. Similar to skilled movement being based off a strong foundation/hierarchy of mobility, stability, and controlled mobility patterning, it will be important to analyze how myofascial trains may also relate to this chain of command. Just like how most non-pathologic upper extremity movements are often preceded by core contraction, or hand movements are preceded by shoulder activity, a train/movement that is considered more for skill, complex (tri-planar positioning), or highly coordinated CPG activity (large degrees of freedom or large velocity/acceleration) should be preceded by more foundational trains.

This hierarchy of activation can be seen at more fundamental levels, such as within muscles. Typically when movement is required, type I motor units are activated before the type II units. To bypass this hierarchy, selective recruitment, which is an ability to inhibit lower threshold neurons so the higher threshold motor units may activate more readily, may be achieved using the garment. If selective recruitment is desired, the tension lines may be placed to potentially pre-activate the lower threshold nerves so the next step in the recruitment pattern, the type II motor units, are expedited. Understanding the physiology associated with loading is important because wear time of the garment, depending on effect desired, may be a more chronic timeframe with type I muscle fiber adaptation, verses short wear requirements for fast twitch adaptation.

Following the appreciation of the myofascial planes within the body, it is important to determine muscle strength and length deficits contributing to pain or improper posture. Identifying this relationship requires understanding a person's cumulative stress. For example, the instability from lengthened tissue typically occurs in conjunction with an alteration of the stress-strain curve. The changes would show yield and ultimate strength point depression and a shift of the stress-strain curve toward the right. This means that when a tissue is approaching plastic deformation, a large slope, not proportional to the rest of the curve, will be seen. The graphing of a stress-strain curve is important for identifying how an individual adapts or should adapt to the forces they experience throughout the day. Frequently pain, weakness, and length deficits within the body are from an abnormal base of support. Understanding pelvic position, lumbar position, and how the chain is influenced in a superior/distal direction ultimately helps resolve upper extremity, lower extremity, and trunk issues. By fixing the baseline posture, and ultimately neuroarthromyofascial tissue position, one will have changed the firing pattern of the nervous system. The new cortex modulation and rewired central pattern generators may modulate pain, strength, and length deficits.

It is ultimately up to the health care professional to identify true movement pattern deficits verse inert tissue deficits, and their related nature. Analysis techniques may try to identify whether an impairment, such as range or strength truly modifies motion or, rather, if it is the bodies inability to perform and stabilize as a coordinated unit. Often, movement is limited by the inability to stabilize through an available range, thus giving it an illusion of being too tight, too lengthened, or too weak. Selection or customization of the garment benefits from identifying where in the hierarchy of movement the deficit lies. Corrections using the garment may be as fundamental as modifying the posture or as specific as changing muscle/movement firing patterns and/or movement mechanics in a highly skilled activity, such as throwing.

This understanding shows the importance of both generalization and specificity as it pertains to use of the present garment; thus, two embodied categories of the garment have been developed: Generalized Recruitment Modification Designs (GRMD) and Tailored Designs (TD). GRMD will be worn to modify common posturing/movement deficits seen within the public. GRMD requires no prescription, as an individual will be able to categorize themselves with a few different methods. One method will be through an on-line or app questionnaire, which will feed the answered questions through an algorithm identifying appropriate sport/activewear selection. Another method will rely on the consumer's prudence in correlating body type/movement dysfunction with the product description; this method is more empirical in nature. Not all people will benefit from GRMD and this is where the TD becomes important.

TD, in contrast, may be prescriptive. A Tailored Design Professional can be trained to identify the concept of care/enhancement a consumer may require, while also appropriately performing measurements to create a custom garment. The tension lines may be modified to influence certain fascial trains that are in deficit, or the tension lines can be performed to reduce specific/gross symmetrical/asymmetrical muscular weaknesses and myofascial patterning. However, too much input via tension or compression can create problems for the neural modulation and connective tissue/muscular changes; at suitable points of the garment selection and/or customization stages, the amount of force applied by the tension lines to achieve optimal success may be identified. Also, depending on the deficit of motion or posture, the dynamic nature of the tension lines may need to be altered. To influence the more primitive patterns a more global and non-specific symmetrical tension line may need to be used, while to influence a specific skill an asymmetrical and concise tension line may need to be used instead. Using patient perception, muscle timing/neurological activation analysis, and other measurement tools, an asymmetrical sport/activewear line can be achieved to help remedy the asymmetries leading to dysfunctional movement.

The garment may be designed to maximize ergogenic benefits like wicking, muscle recovery, thermoregulation, and proprioception, as well as corrective benefits that will influence factors such as hypertrophy, muscle activation, and posture to name a few. Muscular hypertrophy in particular may be targeted by the garment. The purpose of the following is twofold: (A) to give a brief overview of the hypertrophy process and (B) to summarize how the tension lines in the garment may influence muscle growth.

Hypertrophy is defined as an increase in the size of an existing muscle fibers cross sectional area. This increase in fiber size is most commonly known to be influenced through resistance training. Whether you are an athlete, clinical patient, or just looking to get into better physical condition, increasing lean muscle mass is an important factor in any population that is looking to improve health and performance. This is justified by the strong correlation between muscle cross-sectional area and muscular strength. Increases in skeletal muscle have shown to facilitate improvements in areas such as strength, power, endurance, joint stabilization, and posture. However, prior to a fiber increasing in size and strength, an increase in neural drive must occur via increased neural recruitment, firing rate, timing and pattern of discharge, and a reduction in inhibitory mechanisms.

Once the body has adapted neurologically, hypertrophy becomes the dominant result of continued resistance training. During the process of hypertrophy there is an increase in both the amount and size of contractile proteins actin and myosin within the myofibril, as well as an increase in the number of myofibrils. As the new myofilaments are added to the existing myofibrils, there is an increase in the myofibril diameter culminating in an enlargement of the fiber and associated muscle(s) or muscle group(s). Research indicates that the initial gains for muscle growth are greatest at about two months after the onset of a resistance training program, with the ability to increase lean muscle mass lessening as one's training age increases.

In contrast to currently available corrective garments that only provide acute support on a passive level, the present garment may provide chronic adaptation on an active level. These adaptations may be attributed to factors like enhanced proprioception and muscle activation, which will ultimately influence muscular strength and hypertrophy. The increase in proprioceptive cues is most likely due to improved mechanoreceptor feedback, which may be stimulated by the garment applying compressive forces to the skin. Sensory feedback provides the central nervous system with information in regards to coordination of movements and muscle tone. As positive alterations in such information occur, the body's ability to recruit and contract skeletal muscle will also improve.

The present garment further enhances muscle activation by incorporating tension lines within the fabric that will provide specific degrees of external resistance. This resistance will act against the subject, requiring him to activate the muscles necessary to overcome it. For example, in a subject that has rounded shoulders, the tension lines in a corrective shirt will force the shoulders into a larger degree of anterior rotation. This will result in the subject increasing muscle activation in the underdeveloped posterior muscles in order to pull the shoulders back into proper joint alignment to overcome this resistance. As the subject continues to utilize the shirt as prescribed and his condition improves, the degree of tension within the shirt may be increased. This progressive increase in external resistance will allow for further adaptations in both hypertrophy and strength.

General Design for the Sport/Activewear—Iterations

It is important to note how many different garment designs may be implemented, depending on the desired effect(s) of the garment. Generally, this can be shown through a factorial embodiment and an explanation on the makeup of the embodiment:

If there is tri-planar motion for the shoulder, then the shoulder has three motions that can be “activated” through fabric tension lines.

For each joint there is also the potential to activate ⅓, ⅔, or 3/3 of the available planes of motion with the tension lines. That is, the garment can either have tension lines altering the frontal, sagittal, or transverse plane motion singularly or in greater than one plane of motion.

If, for example, the garment is a shirt with a sleeve that influences the shoulder and further will influence the wrist and hand, the number of planes each joint works through from the trunk to the shoulder to the elbow to the wrist/hand must be determined. Stabilizing the neuroarthromyofascial tissue above the wrist and hand will influence distal patterning, which emphasizes the importance on understanding how each joint interacts.

If the elbow has tri-planar movement, as do the wrist and hand, then the number of possible shirt design combinations, with each joint (shoulder, elbow, and wrist/hand) being influenced in only one plane and not including the trunk, is 3 cubed. This ends up being 27 different possible designs. If, at each joint, planar combinations were allowed, options for “A and B”, “A and C”, “B and C”, and “A,B,C” (A=frontal, B=sagittal, and C=transverse) are added. This means in addition to the three singular tension lines altering only one plane of motion there are now seven combinations at each joint. 7*7*7=343 different possible shirt designs. This did not take into account the trunk, or the pelvic connection with the upper extremity, or the lower leg connection with the pelvis and upper extremity.

Design Concepts for Garment—Fabric

The fabric is essential for the active recruitment component, anchoring the tension lines, providing compression, and stimulating mechanoreceptors. This list is not all-inclusive because as research into fabric development is progressed properties may include, but are not limited to wicking ability, temperature control, biofeedback, and electrical analysis of motor programming and function. As an example of the dynamic ability the fabric has in relation to a certain body segment, fabric selection for a shirt is described. It will be understood that the concepts and application of principles apply equally to design of other garments.

The equation for shirt size will ultimately stem from the strain the shirt undergoes when a person wears the material. Because the shirt will be >3 pieces of fabric sewn together it is important to identify how each fabric deforms under load, thus altering initial non-loaded sizing. The change in length, upon wear, will cause a certain amount of force production imparted on the individual wearing the sportswear. The amount of deformation (length of tension line while wearing the shirt−length of tension line before putting the shirt on=deformation or strain) the tension lines undergo while wearing the article of clothing determines force imparted on the person to achieve proper neuroarthromyofascial balance and recruitment. Studies show it may be safe to use a resistance of less than 3-4% of an individual's body weight to increase max voluntary isometric contraction (MVIC). This percentage was founded more for the hip so it requires testing to ensure appropriate recruitment patterns for each joint and desired effect.

Certain elements of the shirt will create a compression force that may work to activate mechanoreceptors both superficially and intra-articularly, while other elements, specifically the tension lines, are more important for achieving the shirt's primary objective, active resistance. A specific objective clustering may be used to create an algorithm determining what tension is most specific to an individual's needs. If the shirt has the tension lines built into it, then during the process of stitching the fabric into place, the change in deformation with wear must be accounted for. One way to do this is using a tensionometer, or in the initial stages, a crude suitcase-weighing hook. With the measurement device, determine upon what strain 3-4% of 180 pounds is, or rather, 5 to 7 pounds. The change in shirt length at 3-4% of body weight will be used to determine how the material needs to be sewn into the shirt. For instance, if the measurement taken on the human body from the anterior acromion to the xiphoid process is 20 cm, and it was identified for fabric consisting of 84% polyester and 16% elastane a 6 pound force produced 50% strain, then the final product may need to have the material from the anterior acromion to xiphoid process at around 12.5 cm. The fabric complex will differ between body parts and designs. In one example, a garment containing 83% nylon and 17% spandex for the material between the anchors and insertions may be used for the shoulder joint, while the surrounding material may contain 86% polyester and 14% spandex.

Design Concepts for Upper Extremity—Scapular Motion

The scapula is involved with scapulothoracic mechanics and very closely related to glenohumeral joint activity. Scapular motion consists of anterior and posterior tilting (sagittal plane), internal rotation and external rotation (transverse plane), and upward rotation and downward rotation (frontal plane). The glenohumeral motions are flexion and extension (sagittal), abduction and adduction (frontal), and internal rotation and external rotation (transverse). The key muscles targeted in the upper extremity include the serratus anterior, rhomboid, and mid/lower trap muscle groups, but other muscles may be targeted depending upon the specific condition to be addressed. The rhomboids and serratus anterior connect the anterior and posterior portions of the spiral line creating synergy during motions involving oblique force vectors. The purpose of the garment targeting this component specifically, or linking it to the joints above/below, will be to control the abnormal patterns identified. Further, while scapular targeting with an upper extremity garment is described, such a garment may also be configured to address stabilization of the trunk. The key muscles targeted for stabilizing the trunk relate to the cylindrical core, which is a combination of all fascial trains. Certain trains are more involved with isometric type motions, while others are more involved with dynamic mobility.

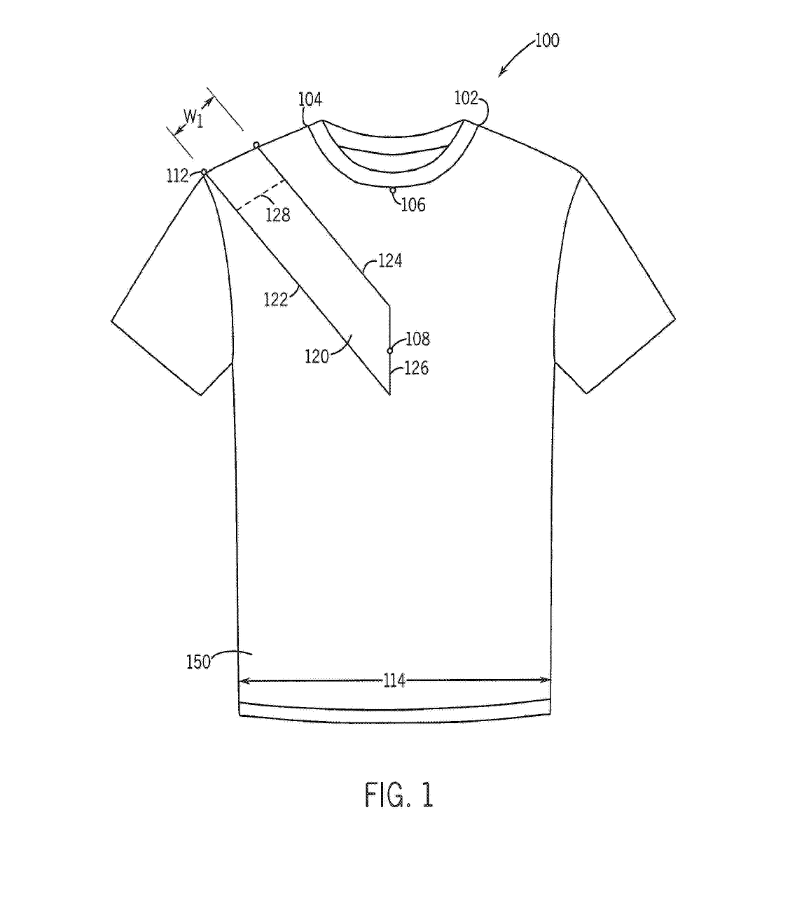

FIG. 1 illustrates an exemplary embodiment of a NEW garment styled as a shirt 100 for enhancing scapular motion. Measurements are provided for positioning a fabric tension line 120 within the shirt 100 so that the shirt 100 protracts, anteriorly tilts, and internally rotates the scapula, thus bringing the scapulothoracic and glenohumeral joints into a non-centrated position. The measurements are taken between anatomical landmarks for purposes of exemplary demonstration; the locations of and distances between critical points may change for both custom-tailored and off-the-shelf designs.

A neck line identifies the appropriate circumferential measurement for the neck. The lateral landmarks for the neck line are the left shoulder vertice 102 and the right shoulder vertice 104. The anterior landmark is the sternal notch 106, and the neck line may be anterior to the sternal notch 106. The posterior landmark for the neck line is not in relation to a specific vertebral segment, but connects the lateral landmarks in the shortest distance.

A peripheral seam 122 and a central seam 124 mark the lateral boundaries of the fabric tension line 120. At the seams 122, 124, the garment transitions between the fabric tension line 120 and the surrounding clothing material 150. The anchor 126 of the fabric tension line 120 is located on the anterior aspect of the shirt 100, approximate the xyphoid process 108. Preferably, a midpoint of the width W₁ at the anchor 126 is positioned over the xyphoid process 108 and the fabric tension line 120 extends inferior and superior to the xyphoid process 108. The elastic material of the fabric tension line 120 pulls toward the anchor 126. The angles upon which the fabric tension line 120 enters into the anchor 126 help determine muscle force pattern. In the upper extremity the fabric tension lines typically will exist obliquely in both an “X” (or frontal) plane and a “Y” (or sagittal) plane, with a vector force emphasizing “X” recruiting more active retraction and “Y” recruiting more active posterior tilt.

The angles are determined by the point at which the fabric tension line 120 crosses the peripheral line between the right shoulder vertice 104 and the right lateral acromion 112. In the illustrated embodiment, the peripheral seam 122 intersects the right lateral acromion 112. The fabric tension line 120 then terminates at the insertion 128 on the posterior aspect of the shirt 100. The fabric tension line 120 descends on the posterior aspect a suitable distance, such as two inches, to provide adequate gripping of the upper extremity. The portion of the fabric tension line 120 on the posterior aspect may include an adhesive or tactile fabric, or another adhesive or securement mechanism, to improve gripping of the upper extremity at the insertion 128.

The width W₁ of the fabric tension line 120 may be uniform or variable along the length. The width W₁ may provide sufficient surface area for the fabric tension line 120 to adequately secure against the wearer as described below. A width W₁ that ascends three inches from the right lateral acromion 112 is an example of a suitable width W₁, with suitability varying according to selection of materials, elasticity, and targeted muscles. Other measurements, such as the overall length of the fabric tension line 120, may similar vary with the materials used, elasticity selected, muscles targeted, or anatomical features or performance variables of the individual. Locations, such as the location of the anchor 126, insertion 128, and peripheral line crossing, may similarly vary. For example, the location of the anchor 126 may change as a better understanding of how the x:y ratio influences motor recruitment develops. Shirts 100 tailored for specific individuals will show significant variance between people because some may require more active recruitment of the lower trap, while others may require more active recruitment of the middle trap. The waist 114 measurement is taken under the iliac crest where the inferior portion of the shirt 100 will grip and prevent the shirt 100 from “riding up.”

The landmarks in this example do not take into account further modification that may be required to promote better active positioning. For example, the shirt 100 may include a second tension line anchoring from the posterior component of the spine, at around T7, and wrapping anteriorly to promote serratus anterior activity. This may be beneficial in how it will act in synergy with the before mentioned mid/lower trapezius active recruitment. Another example modification includes increasing the distance that the tension lines lie from the shoulder vertice. For instance, a modification may be in having this line descend three inches further or be independent of the current line to promote greater glenohumeral joint activity. Greater passive internal rotation, which will cause active external rotation and rotator cuff recruitment, may improve shoulder functional patterning and even improve scapular stability. Furthermore, shoulder, or rather glenohumeral joint, specific tension lines may exist with or without the scapular design. The tension lines may be based around altering rotation for rotator cuff activity. The lines may also alter frontal plane motion by resisting inferior to the GH joint for supraspinatus activity. The force applied by the garment may be selected to create rotator cuff stability, such as supraspinatus activity, while not overactivating the deltoid tissue. The tension lines may also exist to pull the glenohumeral joint into abduction when a person works with the upper extremity in a relatively adducted state. Another option is for sagittal plane recruitment, which, depending on tension line placement, may cause compression forces for stability or stimulate rotator cuff activity.

Design Concepts for the Trunk

The shirt 100, or a different fashioned garment that covers the trunk, may be designed to affect the trunk, or core. The trunk is the base for all movements. Without trunk activity the extremities do not have a sound base to stabilize upon. There is good evidence on natural asymmetries within the trunk, which is why it must be noted whether these are pathologic for a patient or not. Just like the trunk may be influenced into tri-planar motion/positioning, so may the cervical and thoracic spine. The thoracic spine and rib structures may be manipulated by facilitating the diaphragm with the utilization of a tension band circumferentially compressing the lower ribs. The internal or external rib rotation may be influenced with either specific tension lines promoting proper position or facilitating an abnormal position.

Secondary to the rib orientation being largely due to intra-articular forces and passive myofascial tissue tone, this is one body part that may benefit from force vectors repositioning the thoracic cavity. Of course, if realignment is truly sought, then low levels of force creating an active recruitment pattern opposite the postural dysfunction will help to reorient over time. This method of rehab may be beneficial for scoliotic patients. The lumbar spine, though touched on before, deserves revisiting for its similarity to the thoracic and cervical spine. The garment may alter myofascial tension to decrease soreness, stiffness, or pain. If a patient is experiencing significant tone in the erector spinae, or any other myofascial structures having contractile properties, placing the fabric tension lines posterior will help recruit the anterior train of muscles and reduce tone/tension posterior with antagonistic inhibition.

In an embodiment, the tension within a shirt may be modified as it is being worn, and an interval ratio may be prescribed to contract the erector spinae at low levels of neuromuscular activity, followed by anterior chain activation at larger levels of muscle activity. The analysis of CPG patterning and pain homunculus activity will help determine more appropriate ratios most relevant to a patients care. The neurologic concepts used to decrease tension include, but are not limited to, the golgi tendon reflex and antagonistic inhibition, which both occur in the spinal cord.

Because the trunk region has a lower density of proprioceptive nerve fibers, it is important to consider the health of the skin for proper afferent activity. The nervous system's relay of the external environment may depend on more than the health of skin tissue, but also on potential neural mobility within the tissue. Compression by the garment may have implications on an individual's pain/sensitivity. Contrary to popular belief, the reason pain is perceived comes not exclusively from damage to muscles or bones, but rather the sensitization of the nervous system within stressed tissue. As muscles and bones respond to stress, whether it be acute or chronic, tissues in the body changes. These changes might involve protein being released from a cell, or perhaps, the breakup of collagen bonds within connective tissue. Whatever the source, the damaged tissue is not actually causing pain. Instead, the brain decides that these changes are significant enough for a person to be aware of and, in turn, causes an individual to interpret these signals as pain, stiffness or general discomfort.

Design Concepts for Hip and Knee—Valgosity Restoration

The pelvis is a direct link between the lower extremities and the lumbar region. The identification of pelvic position, and the reason for pelvic position, can be very complex and requires identification of both extrinsic and intrinsic forces acting upon the pelvis. For instance, hip position, and thus pelvic position, can be influenced by foot position secondary to a chain reaction. This linkage between foot and hip is shown primarily in weight bearing and will vary from individual to individual. Because certain people remain in weight bearing positions with more or less muscle activity, more or less passive inert tissue tension, and different joint congruencies, the pelvic positioning, secondary to the foot/ankle, may be different within the population. This concept of the foot/ankle determining and dictating proximal joint position and muscle activity is not as accepted as the vice versa, which is hip activity dictating the distal joint positioning. Despite this more widely accepted notion, it is still important to determine how the distal segments affect the proximal. Secondary to the hip being a joint that moves in three planes, there can be compensatory deficits in each plane, which can be addressed individually.

By identifying the primary patterns of movement a person performs throughout the day one is able to identify why and how the tissue remodels. It is important to remember the myofascial trains functionally link muscles and promote stability, so as to improve the economy of stability/motion. But, if certain patterns are over-utilized and rely on specific trains, the myofascial and CPG functional unit become over-emphasized and ingrained during the remodeling process. Disproportion in the body leads to compensation. For instance, if someone utilizes the SFL to a greater extent than normal, and provides it a mechanical leverage through pelvic positioning, this person will find it difficult to adduct through the hip and may compensate either through valgus collapse in the knee with compensated trendelenberg or varus collapse in the knee with uncompensated trendelenberg. This is a brief example, but because each joint has multiple muscles interacting with tri-planar motion people develop many different tissue deficiencies for their movement discrepancies. This wide possibility of movement and motion makes it impossible to list each potential compensation.

With this being said, the garment may be designed to focus primarily on reducing anterior pelvic position by stimulating gluteal recruitment, reducing femoral internal rotation and valgus collapse in the knee by improving hip external rotator recruitment, and reducing hip adduction/abduction either secondary to hip abductor weakness or tightness respectively. Because of the linkage between the pelvis and hip they are often talked about together, and it is thus important to stimulate proper femoral-pelvo or pelvo-femoral position during functional movement. The tension lines can extend in many directions, each force vector exerting different/unique effects from origin to insertion, including but not exclusive to: lumbar region into the pelvis, lumbar region into the hip, pelvis into the hip, pelvis into the lumbar, hip into the pelvis, or hip into the lumbar. For instance, an individual may need greater gluteal control through the hip during functional movement, stemming partially from excessive anterior pelvic tilt and gluteal lengthening. The lengthening reduces the mechanical leverage the gluteal muscles are able to provide during functional movement. Instead of using the tension lines from the lumbar to hip region one may extend the anchor to insertion only from lumbar to pelvis for improved gluteal recruitment and reduction in potential force increasing anterior pelvic tilt.

There are many ways the lumbar, pelvis, and hip interact, and it is important this interaction is analyzed with an appreciation for how adding tension lines may improve one component of movement and impair another. For the lumbo-pelvic-hip region the tension lines may originate proximal to the pelvis, around the iliac rim, ischial tuberosity, or conical muscle mass surrounding the femur. It will be possible to influence the pelvis in an anterior and posterior tilt (sagittal), pelvic drop and elevation (frontal), and anterior and posterior rotation (transverse). The lumbar and hip may be influenced in flexion/extension, rotation, or lateral flexion. The type of contraction the tension lines intend to develop may stem from starting position and how much joint motion is allowed at a joint in a particular direction. For example, if contraction of the obliques in the abdominal region are desired then the vector stimulating this contraction would be oriented primarily in a rotational plane of motion. Because the lumbar spine observes minimal rotation this may create more of an isometric contraction with minimal correcting motion for realignment. This is in contrast to sagittal plane correction, where a spine with grade III joint mobility and abnormal posturing may actually use the tension lines to correct out of the abnormal position into proper posture.

The condition of the knee is a product of proximal and distal forces, joint congruencies, and movement patterns. Identifying the line of gravity in relation to the knee is important in understanding how gravity influences joint positioning and motion. Typically the line of gravity falls medial to the knee joint causing medial joint compartment compression, which is why knee osteoarthritis is typically in the medial compartment. Not everyone presents with medial knee joint problems; this is because the ankle and hip affect positioning as described above. If the hip is weak the knee may be in a valgus position; if the hip shows excessive anteversion the femur may demonstrate an excess in internal rotation to correct the malalignment; if the lumbar spine is laterally flexed contralateral to knee involved it may cause varus positioning, while if it were flexed ipsilateral it may cause valgus positioning. If the ankle has limited inversion during preswing then an increase in tibial rotation may occur causing a predisposal to future injury; if minimal dorsiflexion is allowed during a change to the terminal stance, a hyperextended torque may occur within the knee, and the same may occur if minimal 1^{st} metatarsalphalangeal dorsiflexion is allowed.

The anchor and insertion for the tension lines affecting the knee may both originate and insert proximal to the knee at the lumbar-pelvic-hip region. Or, the tension lines may extend from a proximal region distal to the superior/inferior aspect of the knee. The lines may be singular in plane propagation or may traverse multiple planes in an oblique pattern creating valgus positioning, varus positioning, femoral internal rotation, femoral external rotation, knee extension, and/or knee flexion. Also, the tension lines may originate and insert distal to the knee exerting influence from its location in the foot/ankle region. The tension lines for the garment may not be as parallel when comparing the female and male version of the lower extremity secondary to males and females having different arthrokinemtic positions and myofascial recruitment strategies.

It is further important to understand how the center of mass (COM) affects joint position and muscle recruitment patterns. Body position is a product of anthropometry, joint alignment, passive inert tissue stability, muscle recruitment strategies, proprioception input, etc which feed into an algorithm determining where a bodies center of pressure and mass lie. The COM may alter pattern recruitment strategies. For instance, a person of larger stature and anterior COM displacement, secondary to adipose accumulation, may develop a hip strategy for balance verses someone of smaller stature, which could easily utilize an ankle strategy. Over time, this may alter what muscles remain tonic and where restrictions are seen. This is by no means a perfect example, but gives insight into how people may be affected.

The COM is a summation of many individual centers of mass. When selecting or customizing a garment, it is important to consider which anatomical segment is desired for modification and what that individual segment's COM is. This is found by identifying the primary joint segment involved with the movement pattern to be corrected and calculating how each segment (segment defined as joint to joint) contributes to the COM. Calculating COM facilitates identification of different afferent inputs into the nervous system and potential force vector resistances most appropriate for a body segment to be recruited correctly.

An exemplary garment addressing knee valgosity is described. The valgosity within a knee may exist secondary to multiple underlying issues. Anatomical predisposition, such as alignment within the hip or the knee, may promote diminished muscle activity and reliance on inert tissue. Diseases targeting the myofascial construct may diminish muscle strength and create reliance on inert tissue. Abnormal posturing, in an excessively anterior tilted pelvic position, may elongate the gluteal tissue and reduce their ability to contract and enact a stabilizing force on the hip and knee. The reasoning behind the valgosity deficit can be very vast, thus it is important a health practitioner prescribe a garment suited to a patient's specific desired functional goals.

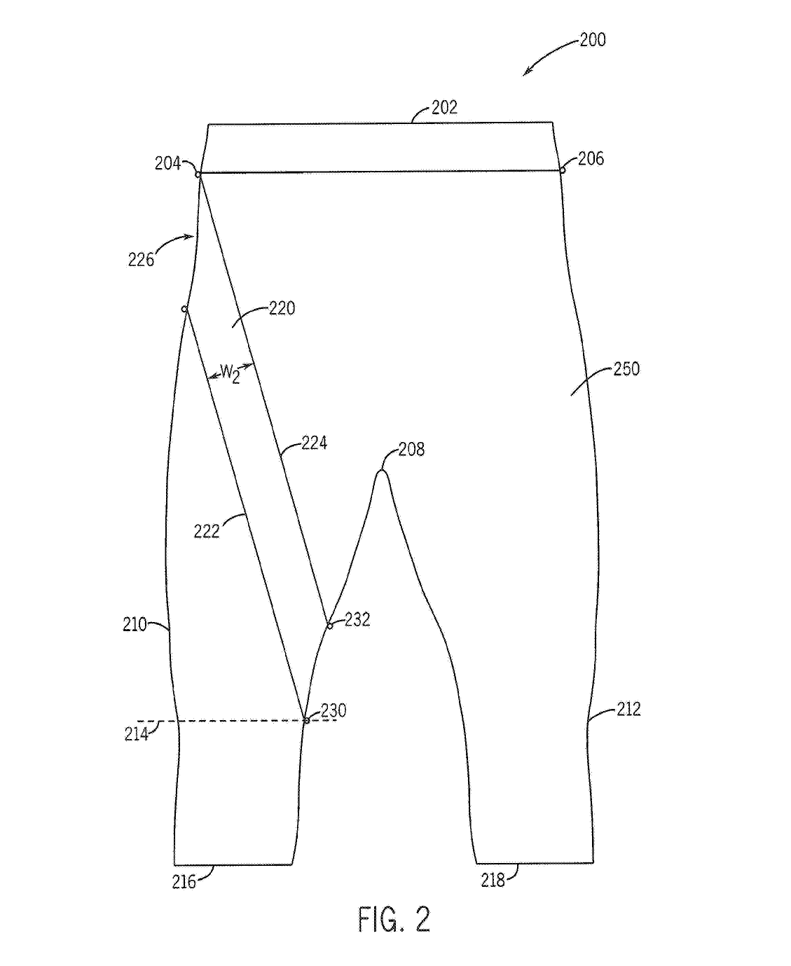

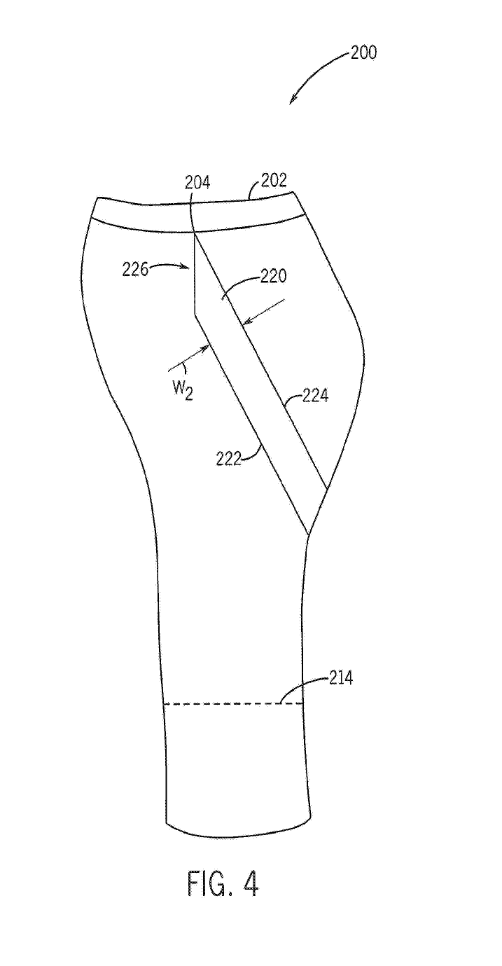

The present garment for a knee application may be fashioned into a pair of pants or shorts, an undergarment, a leg wrap, a thigh-high sock or stocking, or another suitable garment for covering the knee region. The garment may alternatively be incorporated into another garment; for example, the garment may be a pant leg sewn into a pair of pants. FIGS. 2-4 illustrate an exemplary embodiment of the garment as a pair of compression pants 200. The pants 200 may promote the firing of the gluteus maximus, posterior gluteus medius, and adductor tissue (the adductors inserting onto the linea aspera) secondary to the tension lines passively positioning the hip in internal rotation, flexion, and adduction. The passive positioning is a sensory stimulus to activate the more proximal tissues needed for knee support. This design will be beneficial for individuals experiencing valgus collapse during dynamic motion. The amount of tension, in pounds of force, will vary per individual's starting strength, and the design may have different levels of resistance to progress into.

Referring to FIG. 2, the waist 202 may sit two inches above the iliac crest, the left anterior superior iliac spine (ASIS) 204, and the right ASIS 206. The waist 202 may be measured from left ASIS 204 to right ASIS 206. The left pant leg 210 and right pant leg 212 diverge at the gluteal fold 208. The pants 200 extend to mid-calf, inferior to the apex of the gastrocnemius muscle. The cuffs 216, 218 of the pant legs 210, 212 may have an elasticity that anchors the pants 200 at this point on each leg, to resist proximal pull from the fabric tension lines.

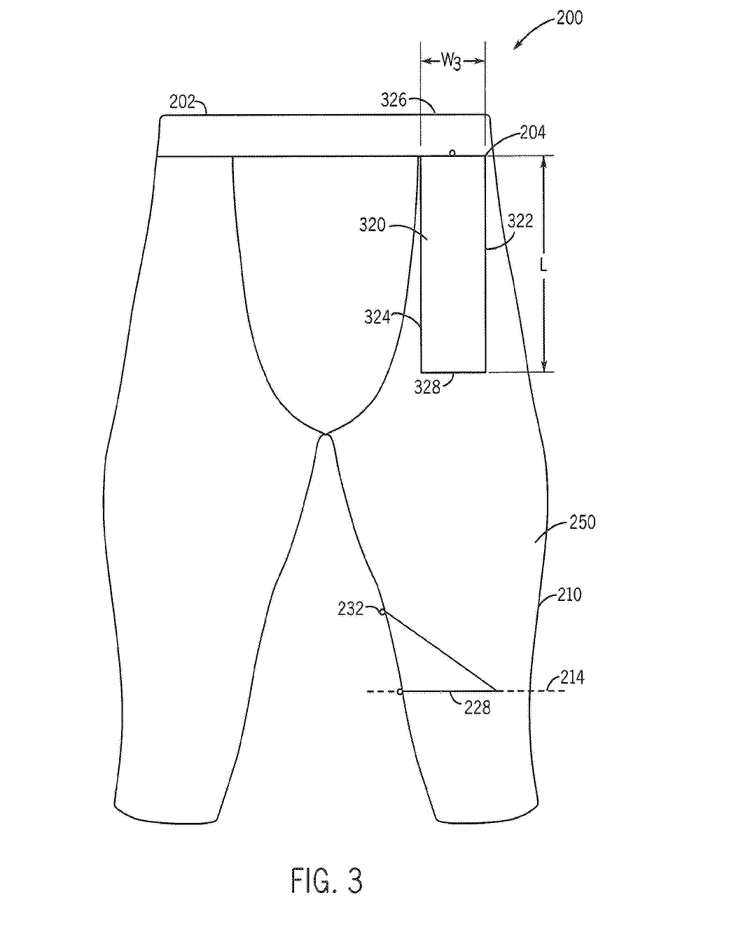

A first fabric tension line 220 extends from the lateral aspect of the pants 200, superior to the hip joint, medially across the posterior aspect of the pant leg 210, descending to the knee joint line 214. A peripheral seam 222 and a central seam 224 define the lateral boundaries of the first fabric tension line 220. At the seams 222, 224, the garment transitions between the fabric tension line 220 and the surrounding clothing material 250. The peripheral seam 222 may intersect the knee joint line 214 at a point 230 on the medial aspect of the pant leg 210. The central seam 224 may extend from the left ASIS 204 to a point 232 superior to the point 230 on the medial aspect of the pant leg 210. As shown in FIG. 3, the central seam 224 may further wrap around to the anterior aspect of the pant leg 210 and descend to the knee joint line 214 while extending laterally across the anterior aspect of the pant leg 210. The peripheral seam 222, in contrast, may terminate at the point 230 and not extend to the anterior aspect or below the knee joint line 214.

The anchor 226 of the first tension line 220 may be positioned over the hip joint approximately level with and posterior to the left ASIS 204. The insertion 228 of the first tension line 220 may be positioned over the knee joint on the anterior aspect of the pant leg 210. The first fabric tension line 220 may cause the hip joint to passively adduct, internally rotate, and extend. To partially or fully counteract, or even overcome, the tension forces that cause extension of the hip joint, a second fabric tension line 320 may be provided on the anterior aspect of the pants 200. In some implementations, the second fabric tension line 320 may have larger (i.e., stronger) vertical elasticity than the first fabric tension line 220, thus causing passive hip flexion to be recruited against for more appropriate gluteal activity. The second fabric tension line 320 descends from the waist 202 to a point on the anterior aspect a suitable distance L from the waist 202 to enact an anterior tilt on the hip joint. An exemplary suitable distance L is five inches. A peripheral seam 322 and a central seam 324 define the lateral boundaries of the second fabric tension line 320. At the seams 322, 324, the garment transitions between the fabric tension line 320 and the surrounding clothing material 250. The peripheral seam 322 may intersect the ASIS (e.g., the left ASIS 204). The anchor 326 of the second tension line 320 may be positioned at the waist 202, with the insertion 328 being located at the opposite, inferior end of the second tension line 320.

The width W_{2,3} of the fabric tension lines 220, 320 may be uniform or variable along the length. The width W_{2,3} may provide sufficient surface area for the fabric tension line 220, 320 to adequately secure against the wearer. For example, a width of three inches is suitable for both fabric tension lines 220, 320, with suitability varying according to selection of materials, elasticity, and targeted muscles. Other measurements, such as the overall length of the fabric tension lines 220, 320, may similarly vary with the materials used, elasticity selected, muscles targeted, or anatomical features or performance variables of the individual. Locations, such as the location of the anchors 226, 326 and insertions 228, 328, may similarly vary. Pants 200 tailored for specific individuals will show significant variance based on individual requirements.

Design Concepts for Elbow—Pronation and Supination

The elbow, like many of the other joints discussed, is tri-planar, but unlike many of the other joints it exists in primarily the sagittal and transverse planes. Often, dysfunction in pronation and supination create abnormal traction forces between the myofascial-tendo-osseous tissue. If a discrepancy, such as in strength or length, exists between the medial and lateral tissue compartments, pathologic movement may occur. The fabric tension lines for the elbow may be based around altering flexion/extension (sagittal), pronation/supination (transverse), and valgus/varus (frontal plane).

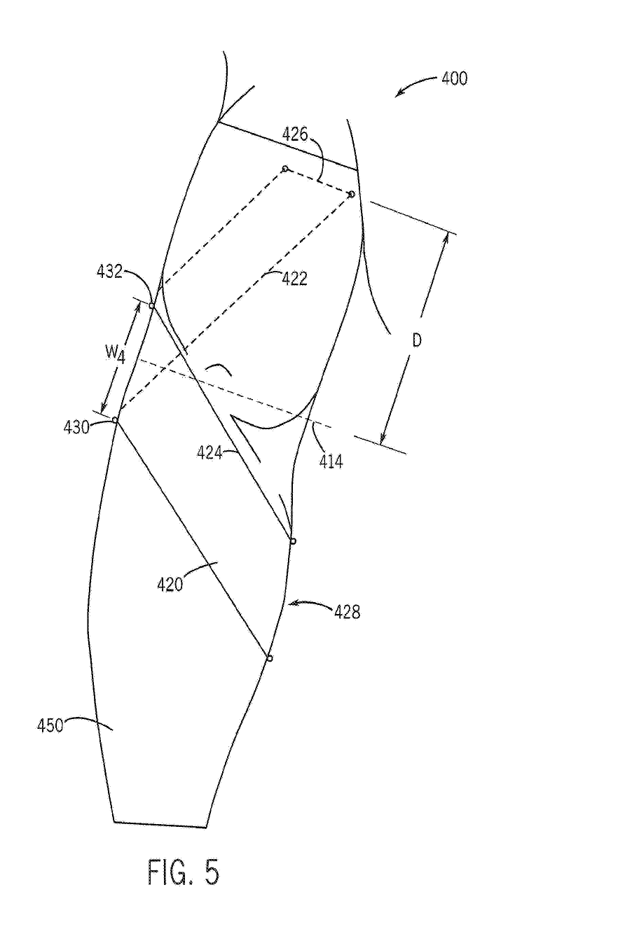

FIG. 5 illustrates an exemplary embodiment of the present garment fashioned into a sleeve 400, although other designs such as an elbow wrap are contemplated. The sleeve 400 is designed to remedy one of the more common issues within the elbow construct, by enacting a change in the amount of pronator muscle recruitment. This design will be of benefit to throwing populations secondary to reduced loading within the ulnar collateral ligament from pronator muscle stabilizing forces at the medial elbow. Also, this design may be beneficial for “tennis elbow” secondary to the force enacting pronator muscle activity reducing inappropriate loading within the elbow.

The end of a fabric tension line 420 that incorporates the anchor 426 may be positioned on the posterior aspect of the sleeve 400 a suitable distance D superior to the olecranon, which aligns with the elbow joint line 414. An exemplary suitable distance D is five inches. A peripheral seam 422 and a central seam 424 define the lateral boundaries of the fabric tension line 420. At the seams 422, 424, the garment transitions between the fabric tension line 420 and the surrounding clothing material 450. The fabric tension line 420 descends toward the elbow, crossing the posterior aspect laterally. The fabric tension line 420 transitions to the anterior aspect of the sleeve 400 when the seams 422, 424 intersect points 430, 432 on the lateral periphery of the sleeve 400. The points 430, 432 may be equidistant from the elbow joint line 414; that is, the elbow joint line 414 intersects the fabric tension line 420 at the lateral aspect at the midpoint of the fabric tension line's 420 width W₄. The fabric tension line 420 continues to descend, crossing the anterior aspect of the sleeve 400 medially until the medial periphery of the sleeve 400 is reached. The insertion 428 is located at this end of the fabric tension line 420.

The width W₄ of the fabric tension line 420 may be uniform or variable along the length. The width W₄ may provide sufficient surface area for the fabric tension line 420 to adequately secure against the wearer. For example, a width of four inches is suitable for the fabric tension line 420, with suitability varying according to selection of materials, elasticity, and targeted muscles. Other measurements, such as the overall length of the fabric tension line 420, may similarly vary with the materials used, elasticity selected, muscles targeted, or anatomical features or performance variables of the individual. Locations, such as the location of the anchor 426 and insertion 428, may similarly vary. A sleeve 400 tailored for a specific individual will show significant variance based on individual requirements. Additionally, the elbow-specific tension lines may be incorporated into a shirt 100 having a scapula and/or shoulder design. If the inadequacy for the elbow is based upon improper proximal stabilization then it will be important the scapula and shoulder design is included. If the tension lines are trying to promote inter-related CPG activity between two joints, muscles, movement patterns, etc the elastic fabric may extend from one joint to another and may not be mutually exclusive in their placement. For instance, an elastic fabric may exist anchoring at the posterior cuff and descending in a vector anterior and medial to the elbow joint line. This type of pattern may be utilized for pitchers having trouble following through during their throwing pattern. If appropriate scapulothoracic mechanics and glenohumeral joint activity are noted then the “upper extremity sleeves” may not be connected with an anchoring shirt and may exist by themselves.

A sleeve 400 may additionally address functions of the wrist and hand. The wrist and hand is complex consisting of many joints. This dynamic anatomy leads to many possibilities in garment design. The wrist, by itself, is more manageable for manipulation than the hand and has three motions that can be altered. Transverse plane alteration may be shown in combination with forearm pronation/supination. Altering accessory transverse plane motion will be performed to elicit mechanoreceptor activity for CPG alteration. Sagittal plane influence will be in association with flexion and extension, while frontal plane will be associated with radial and ulnar deviation. Secondary to the wrist motion occurring in proximity and synergy with the forearm/elbow, the typical design, whether it is a sleeve or shirt, will typically cross both joints. Though this may be a general rule, it is not a law, and some people may benefit significantly from wrist sleeves. This could be utilized for people who spend a lot of time typing and need more appropriate active positioning to reduce soreness, promote joint centration, and allow an increase in endurance. Because supination commonly occurs with extension the tension lines may want to have a vector dorsal to the wrist in an oblique radial line. This will help promote active extension with supination. This serves as an example and is only one component of many that may exist in the wrist.

Design Concepts for Head—Neck Flexor Elongation

The cervical spine consists of eight articulations, including the OA and C7-T1 joints. Often, people have myofascial tone abnormalities with headaches, “stiffness”, and pain existing from the muscle tissue. When this muscle tissue becomes overactive, even in resting posture, the joints can become compressed and neural tissue compromised. In pathologic cervical spines, which may or may not be painful but alter normal scapulothoracic mechanics, the anterior deep cervical neck flexors are typically elongated and weak. By improving muscular activity within the anterior neck flexors, which will help with cervical alignment and reducing posterior tissue tone, an individual may reduce pathologic stress and improve functional ability.

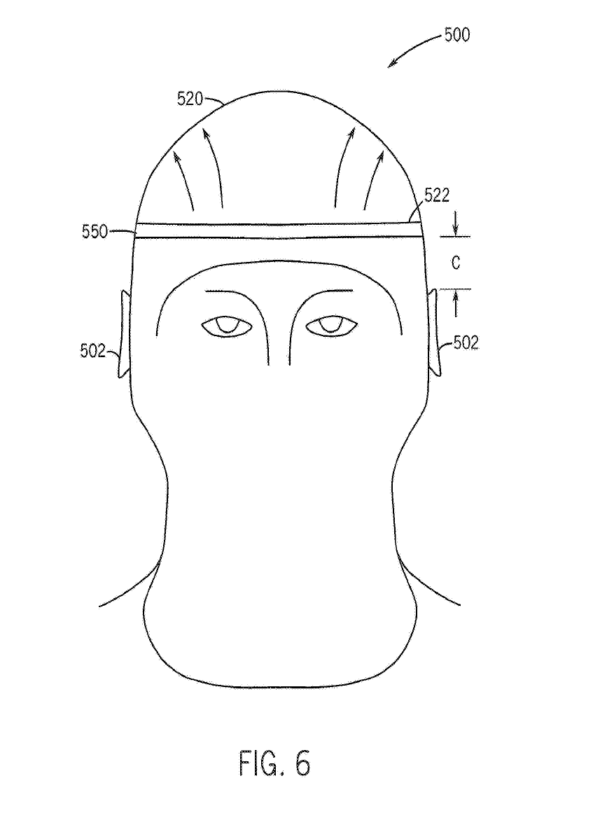

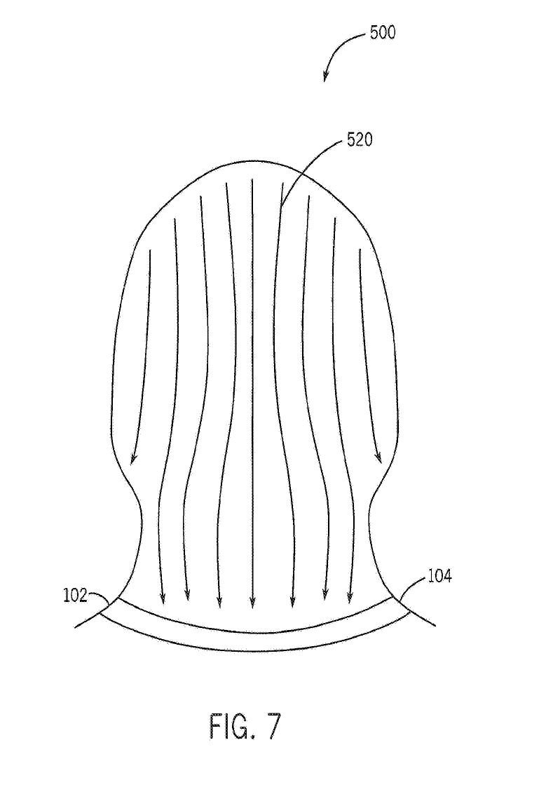

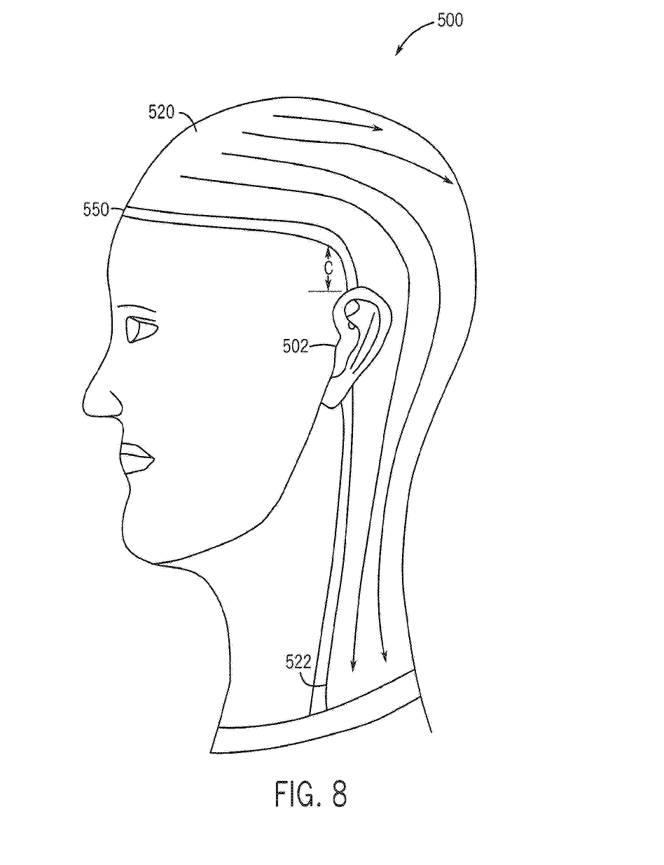

FIGS. 6-8 illustrate the garment fashioned as a hood 500 for activating the anterior deep cervical neck flexors. Beginning a distance C superior to the top of the ears 502, the clothing material 550 may extend around part or all of the circumference of the head. The distance C is selected to allow the hood 500 to grip the head appropriately, and may be about two inches. The clothing material 550 may extend across the front of the head to slightly posterior to the ears 502 on both sides, and then descend to the neck line at or about the shoulder vertices 102, 104. A fabric tension line 520 may be defined by a seam 522 that is offset from and follows the contour of the peripheral edge of the clothing material 550. The fabric tension line 520 may thus extend around the head and cover the entirety of the head and neck superior and medial to the seam 522. As indicated by the arrows on the Figures, the tension force of the fabric tension line 520 pulls from the anterior portion of the hood 500, which includes the insertion, to the posterior portion of the hood 500 at the shoulder vertices 102, 104, which includes the anchor.

As in the other exemplary embodiments, the characteristics of the hood 500, such as circumference, position on the head, position of anchor and insertion, and the like, may be adapted to suit a particular individual with a particular therapeutic need or a particular set of performance criteria. To activate and facilitate cervical spine activity, the hood 500 may extend from the neckline. Alterations of how far anterior the hood 500 is positioned will alter vector torque and influence CPG patterning.

Design Concepts for Foot/Ankle—Eversion/Inversion Control

The foot/ankle complex includes about 33 joints in a tri-planar world, with most myofascial tissue existing in greater than one plane. Despite this multi-planar disposition, the present garment can enact changes in not only multiple, but also singular planes of motion. The plane of motion most highly sought to control in conservative rehabilitation is the frontal plane. This is because many of the muscles crossing medial and lateral to the subtalar joint have vector lines exerting their primary stabilizing force in the frontal plane. While the exemplary embodiment focuses on frontal plane myofascial control, the garment tension lines may additionally or alternatively align with vectors existing in multiple planes of motion. The main joints manipulated by the garment may include, but are not exclusive to, the talocrural, subtalar, talonavicular, calcaneocuboid, and first metatarsalphalangeal. If more stability was sought in the midfoot, and the tibialis posterior was weak, a tension line creating passive eversion, dorsiflexion, and abduction range of motion may be used to recruit the tibialis posterior. Because the body relies on the foot/ankle proprioceptive function for helping to control joint reaction forces within and proximal, the garment may be used to perform this function.

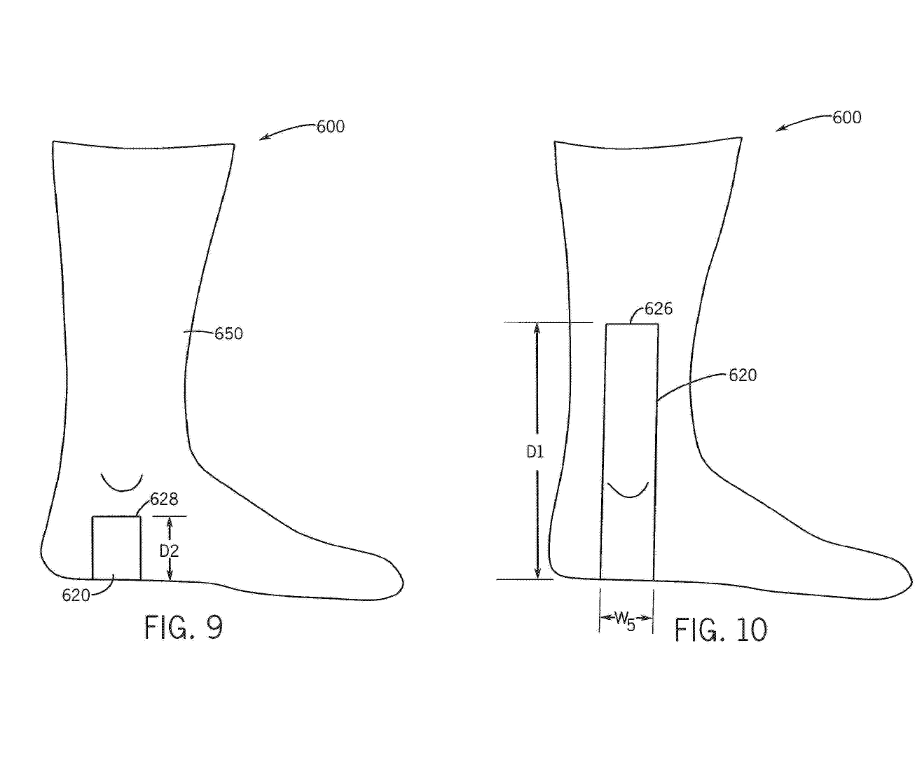

FIGS. 9 and 10 illustrate an exemplary embodiment of the garment fashioned as a sock 600 or stocking, although other embodiments such as an ankle wrap are contemplated. A fabric tension line 620 of the sock 600 is integrated with or attached to clothing material 650, and is positioned to provide torque that places the ankle into eversion, activating the tibialis posterior, flexor halluces longus, and flexor digitorum longus. The fabric tension line 620 may be aligned with the ankle joint, beginning a suitable distance D1 superior to the base of the foot on the lateral aspect of the sock 600, descending to and then wrapping medially around the base of the foot. The fabric tension line 620 then ascends the medial aspect of the sock 600 and terminates a suitable distance D2 superior to the base of the foot. A suitable distance D1 extends sufficiently above the lateral malleolus to provide the necessary torque on the ankle joint. Seven inches above the base of the foot is an exemplary suitable distance D1. A suitable distance D2 provides sufficient gripping of the medial aspect of the foot, but does not impinge on the medial malleolus. An exemplary suitable distance D2 is two inches superior to the base of the foot.

The anchor 626 may be positioned at the end of the fabric tension line 620 on the lateral aspect of the sock 600, while the insertion 628 may be positioned at the end of the fabric tension line 620 on the medial aspect of the sock 600. In another embodiment for placing the ankle into inversion, the distances D1, D2 and the locations of the anchor 626 and insertion 628 may be reversed from the illustrated embodiment. This has the effect of activating the peroneal tissue.

The width W₅ of the fabric tension line 620 may be uniform or variable along the length. The width W₅ may provide sufficient surface area for the fabric tension line 620 to adequately secure against the wearer. For example, a width of two inches is suitable for the fabric tension line 620, with suitability varying according to selection of materials, elasticity, and targeted muscles. Other measurements, such as the overall length of the fabric tension line 620, may similarly vary with the materials used, elasticity selected, muscles targeted, or anatomical features or performance variables of the individual. Locations, such as the location of the anchor 626 and insertion 628, may similarly vary. A sock 600 tailored for a specific individual will show significant variance based on individual requirements.

Varying levels of compression and elasticity will be provided to optimize neuroarthromyofascial recruitment. Recommendations for elasticity will be based on research and empirical evidence. Certain performance variables will be used to determine elastic levels required of an individual to achieve optimal performance. Compression levels may be selected in light of the visco-elastic properties of the tissue to be covered and/or manipulated by the garment. A stress-strain curve with appropriate understanding of the tissue loading, as described above, may be used to identify the proper compression levels.

Monitoring of the sensory homunculus and primary motor cortex will be studied during wear to determine influence on motor function and timing. Devices are currently on the market to measure specific brain waves looking at neural fatigue. Devices similar to the OmegaWave might be utilized to determine appropriate neurological activity during wear of the garment.

Recommendations of wear time will be provided for an individual based on the results of the neuroarthromyofascial performance variables. The sportswear will cause fatigue as the temporal component increases during singular or repetitive sessions; thus, further understanding will be developed with respect to which factors most effectively determine when the positive effects of the performance wear are overridden by negative attributes. More effectively defining this transition will help decrease the risk for potential injury.

Because the understanding of the NEW neuroarthromyofascial physiologic response from the sportswear is driven by inferential conclusions that parallel the intended purpose of the NEW, future research may further enhance an individual's performance from the sportswear. Research may be performed with measures taken to ensure sound methodological design. It will be understood that the concepts are foundationally enabled herein, and the purpose of continued research is to ensure optimal performance gains.

Sportswear is not utilized to indicate specific patient population, but rather the purpose of the design. This product may be utilized by all individuals (not just athletes), but certain variables of each patient's neuroarthromyofascial system must be understood before its allocation into either the patient's activities of daily living OR sporting performances. The design may be used either in preparation for a task/sport OR during the performance of the task/sport. Different recommendations will be made per individual and reason for use.

Exemplary Evaluation for Functional Movement Deficits

As indicated above, in tailored-design settings, the subject is evaluated to determine the optimal parameters for the garment. The evaluation should take into account the neuroarthromyofascialskeletal system. The following is a sample evaluation process and enables but is not limiting of the collection of sufficient information from the subject to create or select the appropriate garment.

Understanding that pain, the threshold associated with pain, and CPG patterning originates from the neurological system is important.

It is important to first identify motion/postures making up >50% of cumulative torque experienced throughout the day. Taking these movements/positions pertinent to an individual and determining where compensation exists will be important for progressive care. After identifying where in the hierarchy of movement the deficit lies, it is important to test strength and range of motion (ROM) capable of making this movement successful.

Posture is not always the elemental component to rehabilitation, and thus evaluation should determine where in the hierarchy of movement compensation exists.

The following could be combined with a journal point system to determine amount of cumulative torque an individual experiences over time. This could help an individual be aware of daily activity, and how much is too much or not enough.

1) Primary Movement (most pertinent):
a) Breakout of movement (one or more)—the breakouts should dictate a progressive exercise routine. They should also be identified for movement compensation, hypertonic locations causing neurogenic claudication, etc, thus palpation and visual inspection is key.
   (a) Range of motion
      (i) Consistency (joint endfeel, etc.):
      (ii) Degrees:
      (iii) neurologic segment associated with stretch:
   (b) Fascial tension lines
      (i) Type of fascial tension:
      (ii) Nerve roots associated:
   (c) Muscle strength
      (i) type of contraction (eccentric/concentric)
      (ii) at what point in the range:
      (iii) neurologic segment associated with testing:
   (d) Nerve tension test associated with movement
      (i) Deficit:
      (ii) No deficit:
      (iii) Palpation to peri-neural tissue:
   (e) Nerve sensitivity
      (i) Spinothalamic
      (ii) Dermatome (sensory)
      (iii) Clonus
      (iv) Proprioception
      (v) Balance
   (f) Sympathetic nervous system
      (i) Omega wear
      (ii) Heart rate
      (iii) Blood pressure
         2) After function, and an understanding of an individual's breakout is determined, it is important to determine sensitivity either from swelling or trigger/latent points.
a) swelling:
   lymphatic distension:
   circumference measurement:
b) trigger points: (using pinwheel is there an inflammatory response—“scratch test”)
c) latent points:
   3) Posture (Needed for ROM): Baseline posture is important to determine how the body compensates.
   4) Desensitization: will they respond to desensitization movements, which is progression into area of impairment or movement progressed to reduce sensitivity further.
   5) General manual therapy hierarchy for massage:
a) rhythmic mobilization
b) effleurage
c) nerve gliding
d) strain/counterstrain (S/C)
e) (S/C) with neurologic principles (NP)
f) Deep Tissue Massage (DTM) with S/C
g) DTM with S/C and NP
h) Active Release Therapy (ART)
i) ART with dense connective tissue palpation
j) ART with muscle palpation
Manual therapy adjunct (may fit between any of the above principles)
a) Mobilization
b) Manipulation
c) TDN
d) TDN with e-stimulation.
   Not everyone needs to start at baseline as many functional screening concepts infer. This is not how the CPG patterning integrates. It is important to break down an individual's current pattern to the most elemental form to determine integration.

This evaluation is not performed over one treatment session, but rather over multiple.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Finally, it is expressly contemplated that any of the processes or steps described herein may be combined, eliminated, or reordered. Accordingly, this description is meant to be taken only by way of example, and not to otherwise limit the scope of this invention.

What is claimed is:

1.-15. (canceled)

16. A method of making a neuroarthromyofascial enhancement garment, the method comprising:
determining a recommended elasticity for an individual;
creating one or more fabric tension lines having the recommended elasticity and having a first end and a second end, the elasticity pulling the second end toward the first end;
forming a clothing material into a shape that fits over a region of the individual containing one or more targeted muscles; and
incorporating the one or more fabric tension lines into the clothing material to form the garment, the one or more fabric tension lines being positioned such that, when the garment is worn by the individual, the one or more fabric tension lines create torque that directs the individual into an abnormal position that causes, in the individual, a neurologic cueing to activate one or more of the one or more targeted muscles.

17. The method of claim 16, wherein determining the recommended elasticity for the individual comprises:
determining a value for each of one or more system variables pertaining to the neuroarthromyofascial system of the individual;
determining a value for each of one or more performance variables related to one or more of the one or more targeted muscles, a CPG, and a movement pattern; and
determining the recommended elasticity from the values of the one or more system variables and the values of the one or more performance variables.

18. The method of claim 17, wherein determining the recommended elasticity for the individual further comprises determining, in the individual, one or more fascial connections associated with the neurologic cueing of the one or more targeted muscles.

19. The method of claim 18, wherein incorporating the one or more fabric tension lines into the clothing material comprises positioning a first of the one or more fabric tension lines such that the first fabric tension line directs a joint of the individual out of a first position into the abnormal position, wherein the first position is one of:
a centrated position, the abnormal position being a desired non-centrated position; and
a non-centrated position, the abnormal position being further abnormally aligned than the non-centrated position.

20. A method of promoting active neuroarthromyofascial recruitment of targeted muscles, CPGs, or movement patterns in an individual, the method comprising:
identifying the targeted muscles, CPGs, or movement patterns;
determining a value for each of one or more performance variables related to the targeted muscles, CPGs, or movement patterns;
selecting a garment configured to activate the targeted muscles, CPGs, or movement patterns according to the values of the one or more performance variables, the garment having one or more tension lines each:
having a first end and a second end, and further having an elasticity that pulls the second end toward the first end; and
being positioned such that, when the garment is worn by the individual, the one or more fabric tension lines create torque that directs the wearer into an abnormal position; and
causing the individual to wear the garment for a wear time determined from the values of the performance variables.

21. The method of claim 20, wherein identifying the targeted muscles, CPGs, or movement patterns comprises:
determining an abnormal condition of the individual; and
determining, as the targeted muscles, CPGs, or movement patterns, one or more muscles, CPGs, or movement patterns that are associated with the abnormal condition, the wear time and the corresponding elasticity of one or more of the fabric tension lines being selected to cause sufficient active recruitment of the targeted muscles, CPGs, or movement patterns to resolve the abnormal condition.

22. The method of claim 21, wherein determining the abnormal condition comprises identifying a lack of force of one or more external rotators of a shoulder of the individual, the abnormal position comprising a combination of a scapula protraction, an internal rotation, and an anterior tilt of the shoulder.

23. The method of claim 21, wherein determining the abnormal condition comprises identifying a loss of muscle activity in one or more muscles that cooperate to maintain abdominal fluids under pressure.

24. The method of claim 23, wherein the one or more muscles having the loss of muscle activity include the pelvic floor, the targeted muscles include the individual's adductors, and the abnormal position comprises an abduction whereby the one or more tension lies facilitate contraction of the adductors.

25. The method of claim 20, wherein selecting the garment comprises determining, based at least in part on the values of the performance variables, the corresponding elasticity needed to cause each of the one or more tension lines to recruit one or more of: the individual's antagonistic muscle complex, one or more of the individual's muscles surrounding hypermobile joints, one or more fascial train muscles, and one or more myofascial structures associated with the individual's fascia.

26. The method of claim 20, wherein the abnormal position is an abnormal posture, and causing the individual to wear the garment comprises determining, from the values of the performance variables, a duration of the wear time that is sufficient to cause the individual to actively recruit the targeted muscles, CPGs, or movement patterns to return to a normal posture.

27. The method of claim 20, wherein the abnormal position comprises an abnormal alignment of centration of a joint of the individual, and wherein causing the individual to wear the garment comprises determining, from the values of the performance variables, a duration of the wear time that is sufficient to cause the individual to actively recruit the targeted muscles, CPGs, or movement patterns to restore centration of the joint.

28. A method of modifying activity of an individual's neuroarthromyofascial system, the method comprising:
determining a desired correction to a first neuroarthromyofascial activity of the individual;
identifying one or more targeted muscles of the individual that are associated with the first neuroarthromyofascial activity;
determining, based on the first neuroarthromyofascial activity and the desired correction, a value for each of one or more variables related to the one or more targeted muscles;
selecting, based on the one or more variables, a garment having one or more tension lines that cooperate to, when the garment is worn by the individual, direct the individual into an abnormal position and provide a resistance that the individual must overcome, by recruiting a second neuroarthromyofasical activity associated with the one or more targeted muscles, to move to a normal position;
determine, based on the values of the variables, a wear time that produces the desired correction; and
causing the individual to wear the garment for the wear time.

29. The method of claim 28, wherein determining the desired correction comprises identifying an abnormal condition of the individual, the desired correction being an alleviation of the abnormal condition.

30. The method of claim 29, wherein the abnormal condition is abnormal posture, and determining the value for each of the one or more variables comprises determining a corresponding elasticity of each of the one or more tension lines such that the individual overcomes the resistant to move to a normal posture.

31. The method of claim 29, wherein identifying the abnormal condition comprises identifying a hypermobile joint of the individual, and the abnormal position is an abnormal alignment of a centration of the hypermobile joint.

32. The method of claim 28, wherein the second neuroarthromyofascial activity comprises activation of one or more of: the one or more targeted muscles, a first muscle group containing the one or more targeted muscles, a second muscle group not containing the one or more targeted muscles, a CPG pattern, and a movement pattern; and
wherein determining the value of each of the one or more variables comprises selecting an elastic tension for the one or more tension lines that causes the one or more tension lines to provide the resistance.

33. The method of claim 28, wherein selecting the garment comprises determining that the second neuroarthromyofascial activity is a desired neurologic cueing that activates the targeted muscles to cooperate in a functional movement.

34. The method of claim 28, wherein selecting the garment comprises determining that the second neuroarthromyofascial activity is a desired neurologic cueing that activates the targeted muscles individually.

35. The method of claim 28, wherein selecting the garment comprises determining an ability of the garment to both facilitate and inhibit neuronal activity in the neuroarthromyofascial system of the individual, such that one or both of the first and second neuroarthromyofasical activities:
occur during one or both of the individual putting on and taking off the garment; and
are selected from the group consisting of: muscle facilitation, muscle inhibition, movement pattern facilitation, movement pattern inhibition, centrally directed pain receptor input facilitation, and centrally directed pain receptor input inhibition.
